(19) United States
(12) Patent Application Publication
(10) Pub. No.: US 2010/0034768 A1
(43) Pub. Date: Feb. 11, 2010

(54) COMPOSITION AND METHOD FOR TREATMENT OF INSECT BITES

(76) Inventor: Miguel A. Castillo, Hilton Head Island, SC (US)

Correspondence Address: WILLIAM H. EILBERG, 316 CALIFORNIA AVE. #785, RENO, NV, 89509, US

(21) Appl. No.: 12/187,950

(22) Filed: Aug. 7, 2008

(51) Int. Cl. A61K 31/74; A61K 35/64; A61K 31/01; A61K 31/215; A61P 17/02

(52) U.S. Cl. 424/78.06; 424/539; 514/762; 514/529

(57) ABSTRACT

A composition for treating wounds of the skin, such as those caused by the bites of small insects or jellyfish, includes a wax or resin, an emulsifier, and ammonium hydroxide. The composition is applied to the skin in a thin layer, and when it dries, it forms a thin, protective film over the wound. Application of the composition has been found to provide temporary and immediate relief of pain and/or itching. The protective film shields the wound, and therefore aids in healing. The composition can be made of any of a variety of waxes or resins, and the emulsion can be formed with long-chain alcohol or fatty acid soaps, such as oleic acid or triethanolamine.

### BACKGROUND OF THE INVENTION

The present invention includes a composition and method for treating minor wounds of the skin caused by the bites of small insects or jellyfish. The invention provides temporary relief of pain and itching, while promoting healing.

It has been known to treat skin wounds caused by the bites of small insects or jellyfish with products that are similar to a clear nail polish. Such products generally contain volatile and flammable solvents, as well as other materials that serve to form a protective coating. An example of such a product is the material sold under the trademark Chigarid, which is available from Colgin Companies, of Dallas, Tex.

The present invention provides a composition and method for treatment of wounds caused by bites of small insects, such as chiggers or mites, as well as the bites of jellyfish. The invention avoids the use of volatile or flammable solvents. The non-flammable material used in the present invention has been found to work well in relieving pain and itching, and in promoting healing.

### SUMMARY OF THE INVENTION

The present invention comprises a composition and method for treating a minor wound of the skin, caused by the bite of a small insect or jellyfish.

The composition includes a wax or resin, an emulsifier, and ammonium hydroxide. If a wax is used, the wax may be selected from the group consisting of carnauba wax, paraffin, microcrystalline wax, bees wax, montan wax, candelilla wax, shellac, or any combination of the foregoing. If a resin is used, it may be selected from the group consisting of hydrocarbon resins, rosin esters, modified rosin esters, phenolic resins, terpene phenolic resins, oxidized hydrocarbon resins and waxes. Instead of the wax or resin, one could use polyethylene or modified polyethylenes including maleic and oxidized modifications.

The preferred emulsifier is oleic acid and morpholine. In general, the emulsifier can include a long-chain alcohol or a fatty acid soap.

The ammonium hydroxide is provided in an aqueous solution which is mixed with the other components.

The composition of the invention is substantially free of flammable solvents. That is, upon application of sufficient heat, the composition will boil before it will burn.

The composition of the present invention is applied in a thin layer over the wound. The composition is preferably applied in a layer which is thinner than that of a typical cream, of the order of 0.125 inches or less. When the composition dries, it forms a thin protective film over the wound. Application of the composition has been found to provide temporary and immediate relief of pain and/or itching. The protective film shields the wound, and therefore aids in healing.

The present invention therefore has the primary object of providing a composition and method for treating minor wounds of the skin caused by bites of small insects or jellyfish.

The invention has the further object of providing a composition for treating insect and jellyfish bites, without requiring the use of volatile organic solvents, or other flammable materials.

The invention has the further object of providing a composition for treatment of insect and jellyfish bites, which composition can be safely and conveniently stored in plastic containers.

The invention has the further object of providing a method of treating wounds caused by insect and jellyfish bites, wherein the method provides temporary but immediate relief of pain and/or itching, and wherein the method also promotes healing of the wound.

The invention has the further object of providing a method of treating wounds caused by insect and jellyfish bites, wherein the method uses a material that is not likely to be abused by those seeking to become intoxicated.

The reader skilled in the art will recognize other objects and advantages of the present invention, from a reading of the following detailed description of the invention, and the appended claims.

### DETAILED DESCRIPTION OF THE INVENTION

The present invention comprises a composition and method for treatment of wounds, caused by the bites of small insects or jellyfish.

The composition of the present invention is an emulsion formed with a wax or resin, the composition also containing ammonium hydroxide. It has been found that the composition of the present invention can be used as a skin medication to treat skin wounds, such as those caused by the bites of small insects or jellyfish. The composition dries to form a substantially water-resistant, thin, protective film over the wound.

Preferred embodiments of the composition of the present invention are described in the following Examples.

### EXAMPLE 1

In this Example, unless otherwise indicated, the percentages of the components are by weight. Each stated percentage refers to the weight of a particular component as a percentage of the weight of the total composition.

Part A

The following components were combined, and heated until melted:

Carnauba wax NC #3 (13.56%)

Oleic acid (2.40%)

Trisodium phosphate (0.27%)

Morpholine (2.72%)

Part B

A quantity of water was brought to a boil, having a percentage by weight of 25.61%, and the mixture of Part A was added to the boiling water. Then, the following component was immediately added:

Ammonium hydroxide (28% concentration) (0.40%)

The composition was mixed for one minute, and then cold water was quickly added, in a percentage by weight of 35.04%, while the mixing was continued. The mixing was performed until the product was uniform, and the mixture was allowed to cool to room temperature.

Part C

A quantity of water, in a percentage by weight of 15.01%, was heated to about 170-180° F. To this water was added ammonium hydroxide (28% concentration, in an aqueous solution) in a percentage by weight of 1.02%. A modified rosin ester (pentaerythritol ester of maleic anhydride modified wood rosin), in a percentage by weight of 3.97%, was sprinkled into the mixture, and dissolved with further mixing. This mixture was allowed to cool to room temperature. The mixture was then mixed with the product of Part B, and water was added to bring the final solids to 20%.

### EXAMPLE 2

Example 1 was repeated, with the addition of an antibacterial agent, in the amount of 0.2% by weight. The preferred antibacterial agent is sodium hydroxymethylglycinate.

### EXAMPLE 3

Examples 1 and 2 were repeated, with the addition of a waterborne perfume. The preferred perfume is waterborne mint, added at a percentage of 0.5% by weight.

The percentages given in the above Examples can be varied. The following table shows the actual percentages used in Example 1, and also shows a preferred range of percentages for each component.

| | Actual % | Preferred Range |
|---|---|---|
| Carnauba Wax NC#3 | 13.56 | 10-20 |
| Oleic acid | 2.40 | 1-5 |
| Trisodium phosphate | 0.27 | 0.1-1.0 |
| Morpholine | 2.72 | 2-4 |
| Boiling water | 25.61 | 20-30 |
| (in Part B) | | |
| Ammonium hydroxide | 0.40 | 0.1-1.0 |
| (in Part B) | | |
| Cold water | 35.04 | 20-40 |
| (in Part B) | | |
| Hot water | 15.01 | 10-20 |
| (in Part C) | | |
| Ammonium hydroxide | 1.02 | 0.5-2.0 |
| (in Part C) | | |
| Modified rosin ester | 3.97 | 2.0-5.0 |

It will be noted that the percentages in the first column of the above table, representing actual percentages used in Example 1, sum to 100.

In place of carnauba wax, one may use petrolatums, hydrocarbon waxes, hydrocarbon resins, rosin esters, modified rosin esters, phenolic resins, terpene phenolic resins, oxidized hydrocarbon resins and waxes, polyethylene and modified polyethylenes including maleic and oxidized modifications.

In the Examples given above, the wax used was Carnauba NC #3. The number three in the latter designation indicates the grade of product, which is light yellow in color. Other grades have different colors, and could be used instead.

Other materials which can be used in place of the carnauba wax include paraffin, microcrystalline wax, bees wax, montan wax, candelilla wax, shellac, or similar types of wax, or any combination of the foregoing.

In place of morpholine, it is possible to use various other amines and also fatty acid amides.

In the above examples, the emulsifier comprises the oleic acid and the morpholine. These substances combine to form the soap which emulsifies the wax. In general, the waxes described above, which are insoluble in water, are emulsified with long-chain alcohol or fatty acid soaps, which reduce the surface tension at the interface of the suspended wax particles because of the solubility properties of their molecules. The combination of oleic acid and morpholine is preferred, because the morpholine provides the desired volatility so that it evaporates from the film and thus renders the emulsion irreversible.

Instead of the oleic acid, one could use other materials to form the emulsion, such as triethanolamine, or any other materials that form a water/wax emulsion. This allows the waxes to remain in a stable solution or emulsion. The invention is not limited to the specific examples given above.

The use of the trisodium phosphate is optional; this component could be omitted.

The function of the ammonium, present in the form of an aqueous solution of ammonium hydroxide, is to stabilize the pH and therefore stabilize the emulsion. For stings and bites which are generally of an acidic nature, the ammonium serves to neutralize the acid associated with the sting or bite, and reduce further burning or damage.

In place of the modified rosin ester, any of the previously mentioned replacements for the carnauba wax could be used. Also, it is possible to use combinations of the ingredients, rather than just one ingredient, to replace another. In each case, the procedure for making and applying the composition would remain essentially the same.

The composition of the present invention may be applied to wounds caused by the bites of any of a variety of small insects, such as chiggers or red bugs, mites, sand flies, fleas, or other small insects. It is also useful on wounds caused by the bites of jellyfish.

The composition of the invention, when applied, has been found to provide immediate and temporary relief of pain and itching. The product forms a protective film over the wound, and therefore promotes healing.

The composition of the present invention is preferably applied to the skin in a thin layer. Preferably, this layer is thinner than a typical layer of a skin cream, and may be 0.125 inches thick or less.

When the composition of the invention is applied to the wound, and when the water in the emulsion evaporates, the wax or waxes are left in a coherent water-resistant smooth film which covers the wound. This film prevents or stops itching, protects the wound, and therefore aids in healing.

A feature of the emulsion formed by the present invention is that it not only seals the active ingredient, but it puckers the skin, pulling the cut together, or, in the case of a burn or other wound, encapsulates the wound. In both cases, it helps to eliminate pain, and assists healing in a significant way.

In Example 3, above, the perfume used was mint. Alternatively, one could use wintergreen, or some other substance. Both the wintergreen and mint fragrances tend to neutralize the smell of the ammonium. Moreover, the wintergreen and mint also tend to provide a cooling effect, providing additional and immediate relief from pain caused by a burn or bite. It is therefore preferred to provide a perfume which also comprises a cooling agent.

The present invention completely eliminates the problems associated with volatile and flammable solvents, by using inert waxes and a soap-based emulsification system. As used in this specification, a non-flammable solvent means a material which, upon application of sufficient heat, will boil before it burns. Among other things, due to its inertness, the composition will not readily react with other materials, including container materials, and therefore can conveniently be stored in plastic containers or bottles.

The composition used in the present invention may be packaged in a small bottle, containing one or two fluid ounces, with an applicator similar to what is used with a bottle of mercurochrome or iodine. The material can be packed in almost any type of plastic bottle. Unlike the case of some organic solvents, it is not necessary to store the composition in a glass container.

The present invention also has an important advantage with regard to public health. As noted above, the products used in the prior art include volatile organic solvents, which are often abused by young persons and others who are seeking to become intoxicated. The composition of the present invention, by contrast, cannot be abused in this way.

The invention can be modified in various ways. The wax used in making the composition can comprise any of the materials listed above, or combinations thereof, and may include other materials not specifically mentioned. The substances used to form the emulsifier can also be varied. These and other modifications, which will be apparent to the reader skilled in the art, should be considered within the spirit and scope of the following claims.

What is claimed is:

1. A composition for treating a wound of the skin, the composition comprising:
a) a material selected from the group consisting of waxes and resins,
b) an emulsifier, and
c) ammonium hydroxide.

2. The composition of claim 1, wherein the material comprises a wax selected from the group consisting of carnauba wax, paraffin, microcrystalline wax, bees wax, montan wax, candelilla wax, shellac, or any combination of the foregoing.

3. The composition of claim 1, wherein the material is selected from the group consisting of carnauba wax, petrolatums, hydrocarbon waxes, hydrocarbon resins, rosin esters, modified rosin esters, phenolic resins, terpene phenolic resins, oxidized hydrocarbon resins and waxes, polyethylene and modified polyethylenes including maleic and oxidized modifications.

4. The composition of claim 1, wherein the emulsifier includes a long-chain alcohol or a fatty acid soap.

5. The composition of claim 4, wherein the emulsifier includes oleic acid and morpholine.

6. The composition of claim 1, further comprising trisodium phosphate.

7. The composition of claim 1, wherein the composition is substantially free of flammable solvents, such that, upon application of heat, the composition will boil before it will burn.

8. The composition of claim 1, further comprising an antibacterial agent.

9. The composition of claim 1, further comprising a waterborne perfume.

10. The composition of claim 9, wherein the perfume also comprises a cooling agent.

11. A composition for treating a wound of the skin, the composition comprising a wax, an emulsifier comprising oleic acid and morpholine, and ammonium hydroxide.

12. The composition of claim 11, further comprising trisodium phosphate, and further comprising an antibacterial agent and a perfume.

13. The composition of claim 11, wherein the wax is selected from the group consisting of carnauba wax, paraffin, microcrystalline wax, bees wax, montan wax, candelilla wax, shellac, or any combination of the foregoing.

14. A method of treating a wound of the skin, comprising applying, to the wound, the composition of claim 11.

15. The method of claim 14, wherein the applying step comprises applying the composition in a layer having a thickness of about 0.125 inches or less.

16. A composition for treating a wound of the skin, the composition comprising:
a) a material selected from the group consisting of carnauba wax, paraffin, microcrystalline wax, bees wax, montan wax, candelilla wax, shellac, hydrocarbon resins, rosin esters, modified rosin esters, phenolic resins, terpene phenolic resins, oxidized hydrocarbon resins, polyethylene, and modified polyethylene, or any combination of the foregoing,
b) an emulsifier, and
c) ammonium hydroxide.

17. The composition of claim 16, wherein the emulsifier comprises oleic acid and morpholine.

18. The composition of claim 16, further comprising trisodium phosphate.

19. A method of treating a wound of the skin, comprising applying, to the wound, the composition of claim 16.

20. The method of claim 19, wherein the applying step comprises applying the composition in a layer having a thickness of about 0.125 inches or less.
